(19) United States
(12) Patent Application Publication
(10) Pub. No.: US 2022/0015860 A1
(43) Pub. Date: Jan. 20, 2022

(54) METHOD AND ARTICLE FOR PROTECTING AIRWAY MANAGEMENT PROCEDURES

(71) Applicant: BUDDHIST TZU CHI MEDICAL FOUNDATION, Hualien (TW)

(72) Inventor: Hsiang-Ning LUK, Hualien (TW)

(21) Appl. No.: 17/226,928

(22) Filed: Apr. 9, 2021

Related U.S. Application Data
(60) Provisional application No. 63/052,517, filed on Jul. 16, 2020.

(51) Int. Cl. A61B 90/40

(52) U.S. Cl. CPC A61B 90/40; A61M 16/0078

(57) ABSTRACT

Provided is an article for protection configured with at least one perforable, modifiable access point positioned to allow airway management procedures. Also provided is a method for maintaining the sterile environment during airway management procedures by covering a subject in need thereof with the article for protection.

### CROSS-REFERENCE TO RELATED APPLICATION

This application claims the benefit of and priority to U.S. Provisional Application No. 63/052,517, filed on Jul. 16, 2020, the contents of which are incorporated herein in their entirety.

### BACKGROUND

### 1. Technical Field

The present disclosure relates generally to an article for protection, and more particularly, to a protective article configured with at least one perforable, modifiable window, aperture or access point, which may be positioned to allow tube, scope or probe for airway management procedures. The present disclosure further relates to a method for maintaining the sterile environment during airway management procedures by covering a subject in need thereof with the article for protection.

### 2. Description of Associated Art

During an infectious disease outbreak, especially those involving the respiratory system, or whenever a patient is in need of airway management procedures in the operation theatre, intensive care unit or emergency room, healthcare professionals carrying out the airway management procedures are often under substantial risk of infection from the splash of sputum, saliva or respiratory secretion of the patient in which an infectious agent may present. Particularly during the pandemic of diseases that involve respiratory syndromes, the need to frequently intubate the suspected or confirmed cases greatly increases the infection risk of the healthcare professionals conducting the intubation.

Although advanced personal protective equipment such as a powered air purifying respirator (PAPR), which is a whole-body coverage suit plus a positive air-purifying respirator, is available for the health professionals, this kind of protective equipment is relatively expensive and poses additional burdens to the wearer. In addition, while the advanced personal protective equipment may ensure maximum protection of the health professionals, it can also limit their visual field and restrain their movements. This can be problematic when a health professional wearing the protective equipment needs to conduct medical procedures that require precise control of hands, such as intubation.

In addition, in places where the resources are limited, or when there is a sudden shortage of medical supply caused by the pandemic situation, the advanced personal protective equipment might not be readily available, but protection of the health professional becomes even more essential. For example, in the pandemic of COVID-19, many airway procedures were conducted with no additional protection adopted (Meng L. et al.; Luo M. et al.; and Chen X. et al.). This greatly increases the infection risk to the clinicians and can further aggravate the situation for the overloaded health care system due to the pandemic.

Recently, a barrier enclosure is reported and tested for protecting clinicians with inadequate access to standard personal protective equipment during endotracheal intubation (Canelli R. et al.). The barrier enclosure reported is a transparent plastic cube called “aerosol box” with two open faces and placed over a patient's head when carrying out the airway procedures. The box is configured with two circular openings on one of the sides allowing the clinician's hands to pass through and operate. However, as it is discussed in the report, such rigid box restricts hand movement and would require additional training of the clinician before use. Furthermore, the weight and the space taken up by the box make transporting and storage difficult. In addition, the box requires cleaning and sterilization after each use, while repeated use of the box might be economical. However, it also increases the risks of contamination to the sterile environment as a result of incomplete sterilization, and also adds workload to the health care personnel.

Therefore, an easy-to-use, easy-to-adapt, convenient, practical and affordable yet effective means for protection is needed to further enhance safety of the healthcare professionals conducting airway management procedures.

### SUMMARY

The present disclosure relates to an article and a method for protection by (1) creation of a shield between the mouth/nose and/or surrounding regions of a subject and his/her surroundings so as to minimize the contamination resulted from the subject's bodily fluid, such as the subject's respiratory secretion; (2) allowing monitoring of the subject by direct visualization of the nose, mouth and/or surrounding regions by, for example, a transparent or see-through sheet or covering or a portion thereof (3) allowing procedures requiring access to the patient's airway such as nose, mouth or other parts of the body by use of windows and/or other apertures, access points or the like designated on the article for protection.

Therefore, the present disclosure is directed to a sizable sheet or covering that is enough to cover the entire head and upper trunk and surroundings thereof of a subject with at least one window or access point through which instrument or a medical device required for the procedures can be passed through without interfering with the operation of the procedures. The sheet or covering is made of a flat, soft, malleable and lightweight material that is waterproof. By placing the sheet or covering over a subject in need of procedures requiring access to his or her airway, operators of the procedures have free and unrestricted hand maneuvers above and outside the sheet. The present disclosure provides an easily accessible and affordable article for protection that is easy to handle and poses little or no interference to the operation of airway procedures.

Another aspect of the present disclosure is a method for maintaining a safe (e.g., sterile) environment comprising using the article for protection as described above.

In at least one embodiment of the present disclosure, there can be at least one (e.g., two or more) windows or access points or the like on the sheet or covering, so that one, two, or more instruments required for carrying out the procedures can be simultaneously or sequentially passed through the sheet or covering through the windows or access points. The position of the window or access point on the sheet or covering has no particular limitation as long as it is designated at the position to allow proper conducting of procedures needed while still maintaining the article's adequate coverage of the head and entire upper trunk of the patient. The windows or access points can be made of the same material as the other part of the article for protection, a different material, or the same material covered with an additional layer of different materials. The windows or access points can be made of a suitable material such that perforations or the like can be made allowing instruments or devices to pass through, while also hindering or stopping the movement of contaminants, such as microbes or other pathogens from the subject receiving treatment procedures. The instruments or devices may be a bag valve mask (an Ambu-bag), ventilator, endotracheal device, video or other stylet/blade for intubation, suction device, and/or any other appropriate device for the procedures. In at least one embodiment of the present disclosure, the first window or access point allows connection to anesthesia breathing circuit to the oxygen mask under the sheet or covering, while the second window or access point allows passage of video laryngoscope, endotracheal tube or Yankauer tip.

In at least one embodiment of the present disclosure, the windows or access points or the like on the sheet or covering are of different sizes to accommodate the passing-through of instruments or devices in different shapes and sizes.

In at least one embodiment of the present disclosure, a precut slit can be configured at the window or access point to allow easy access of the instrument or device. In some embodiments, the precut slit is reinforced with an additional layer of material around the slit. In some embodiments, portions of window or access point may also be reversibly opened or closed to provide access or block passage with a reversible cover configured over the access point. In some embodiments, the window or access point can be configured with a valve that allows inserting of the instrument or device while preventing passing of the bodily fluid from another direction, such as a one-way valve. In some embodiments, a removable cover may be configured at the window or access point to allow inserting of the instrument or device when needed.

In at least one embodiment of the present disclosure, the article for protection is at least one layer of sheet or covering made with any appropriate material that is soft, pliable and lightweight. In some embodiments, the sheet or covering can be two or more layers. The sheet or covering serves as an impermeable barrier to the passage of liquids and at least some gases. Exemplary materials suitable for the sheet or covering may include non-woven and woven fibrous webs, knits, films, foams, polymeric films and other familiar materials that are soft, pliable and lightweight and substantially impermeable to fluid. In some embodiments, a transparent material is desirable to allow for viewing of the underlying subject or medical device. In at least one embodiment, the material has high moisture vapor permeability, but generally impermeable to liquid water so that microbes and other contaminants are sealed out from the area under the covering. One example of a suitable material is a high moisture vapor permeable film such as described in U.S. Pat. Nos. 3,645,835 and 4,595,001, the disclosures of which are herein incorporated by reference. In at least one embodiment, the films may be an elastomeric polyurethane, polyester, or polyether block amide films. These films combine the desirable properties of resiliency, elasticity, high moisture vapor permeability, and transparency. A description of this characteristic of materials can be found in issued U.S. Pat. Nos. 5,088,483 and 5,160,315, the disclosures of which are herein incorporated by reference. Commercially available examples of potentially suitable materials may include the thin polymeric film sold under the trade names TEGADERM (3M Company), OPSITE (Smith & Nephew), etc. Other suitable materials of the sheet or cover include those commonly used in the manufacture of surgical incise drapes (e.g., incise drapes manufactured by 3M Company under the trade names STERIDRAPE, IOBAN, etc.) The materials of the sheet or covering may include, for example, biocompatible polymers, such as permeable, semi-permeable, and/or non-permeable plastic films or combinations thereof, with possibility of meeting N95 or N99 standards as set by FDA (regarding permeability of molecules of given diameter going through the pores). The covering materials may include, but are not limited to, polypropylene, polystyrene, polycarbonate, polyethylene, polyester, polypropene and/or any other biocompatible plastics in sheet or film format or combinations thereof. In exemplary embodiments, the material may also include perforations and/or breathability.

In at least one embodiment of the present disclosure, the article for protection is waterproof or impermeable to liquid so as to substantially provide a shield on the patient and prevent the bodily fluid of the patient from coming in contact with the surroundings.

In at least one embodiment, the article for protection or a portion thereof is transparent or see-through to allow direct visualization of the patient through the article. In some embodiments, the procedure can be assisted with a video-assisted stylet to provide clear visual field for the clinician.

In at least one embodiment, the article for protection is further adapted with a fixing accessory such that the covering can stay in place over the upper trunk of the patient without substantial movement when the clinician is carrying out the procedure. An example of the fixing accessory includes an adhesive tape along the periphery of the sheet or covering. In some embodiments, the sizable sheet or covering can be used without any fixing accessory and is able to stay in place without substantial displacement over the patient.

In at least one embodiment of the present disclosure, the article for protection is configured as a one-piece sheet or film integrally made with a pre-defined line of perforation for tearing or separating. The line is configured to run across the sheet or film and through the window or access point such that the article for protection could be easily teared apart along the line after use while leaving the intubation tube in-place inside the patient. In some embodiments, the article for protection is configured to be made of two or more separate pieces of sheets or films and combined together to form a continuous surface preventing passing or spreading of bodily fluid from the patient. After use, the article for protection can be taken apart to leave the intubation tube in-place inside the patient, while the article for protection can be folded or rolled into separate pieces and discarded.

In at least one embodiment, the article for protection is made of a disposable material that can be discarded after one-time use.

The present disclosure together with the above and other aspects may be understood from the following description of the embodiments of the disclosure and as illustrated in the drawings. The following description, while indicating various embodiments of the disclosure and numerous details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings accompanying and forming part of this specification are included to depict certain aspects of the disclosure. A clearer impression of the disclosure, and of the components and operation provided with the disclosure, will become more readily apparent by referring to the exemplary, and therefore non-limiting, embodiments illustrated in the drawings. It is noted that the features illustrated in the drawings are not necessarily drawn to scale.

FIG. 1 is a front view showing an embodiment of the article for protection of the present disclosure with two areas (shown as rectangles) designated as access points or windows allowing intubation or any other airway procedures or instruments.

FIG. 2 is a front view showing another embodiment of the article for protection of the present disclosure with three areas (shown as rectangles) designated as access points or windows allowing intubation or any other airway procedure or instruments.

FIG. 3 illustrates the transparent article for protection as placed over the patient with one of the access points allowing entry and exit created to accommodate various sizes of endotracheal tubes (ETTs) or video-assisted intubating stylet, with either direct visualization via the transparent article for protection or using a camera (fiberoptic or electrical) in conjunction with the ETT for placement.

FIG. 4A shows a double layered-plastic sheet with two windows or access points marked in rectangles with tape.

FIG. 4B shows the windows or access points marked with crosses and covered with adhesive tapes.

FIG. 4C shows the adhesive tape punctured with the camera module of the video laryngoscope.

FIG. 5A is a photograph showing an embodiment of the article for protection of the present disclosure in use with a facial mask orifice connector.

FIG. 5B is a photograph showing an embodiment of the article for protection of the present disclosure in another use with a laryngeal mask airway tube/connector.

FIG. 5C is a photograph showing an embodiment of the article for protection of the present disclosure in still another use with an intubating stylet/endotracheal tube.

FIG. 6A is a photograph showing an embodiment of the article for protection of the present disclosure in use with a facial mask ventilation. In this embodiment, a plastic sheet is draped on patient's head, face and upper trunk with a facial mask underneath the plastic sheet. Ventilation through the facial mask can be performed easily without air leak.

FIG. 6B is a photograph showing an embodiment of the article for protection of the present disclosure in use with a video-assisted intubating stylet using double-layered plastic sheet. The designated area for access point on the plastic sheet is covered and reinforced with a transparent adhesive tape through which the intubating stylet passes through the article for protection.

FIGS. 7A and 7B show the simulation model of water mist produced by a nebulizer to mimic water droplets caused by coughing or bucking. FIG. 7A shows the spreading of mist in the absence and FIG. 7B shows the spreading of mist in the presence of plastic sheet coverage onto a mannequin's head and upper trunk. An intubating stylet was inserted into mannequin's airway.

FIGS. 8A and 8B show simulation model of water spray to mimic coughing and bucking in the mannequin. FIG. 8A shows the result without coverage of plastic sheet over the mannequin's head and neck, and FIG. 8B shows the result with coverage of plastic sheet over the mannequin's head and neck. Tracheal intubation was performed with intubating stylet technique. Arrows indicate the spreading of the water spray. Imaging was acquired by an infrared thermography machine.

### DETAILED DESCRIPTION OF THE EMBODIMENTS

The detailed description set forth below is intended as a description of the presently exemplified methods, devices and compositions provided in accordance with aspects of the present disclosure, and is not intended to represent the only form in which the present disclosure may be practiced or utilized. It is to be understood, however, that the same or equivalent functions and components may be accomplished by different embodiments that are also intended to be encompassed within the scope of the disclosure.

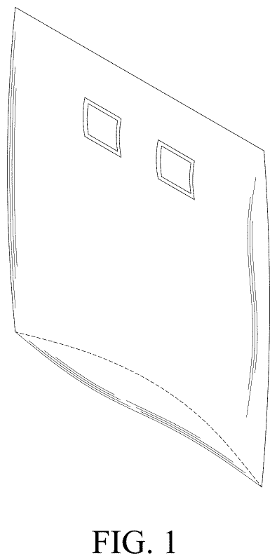

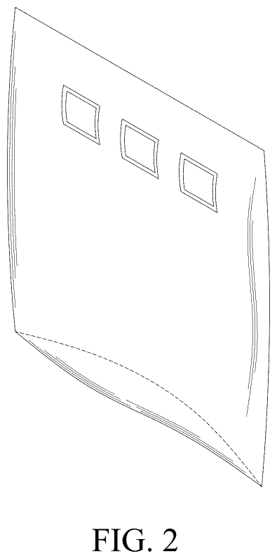

Referring now specifically to the drawings, a transparent and soft sheet for protection according to the present disclosure is shown generally in FIG. 1. The article for protection can be made of a plastic sheet with a size adequate to cover the head and entire upper trunk of a patient. The size of the plastic sheet can be a square of 1 m on each side, or any other shape or size that can efficiently cover the head and upper trunk of a patient. The size of the plastic sheet provides a coverage that could significantly reduce the chance of accidental splash of sputum, saliva, or secretion from the patient onto the surrounding sterile environment or the clinicians in operation during the tracheal intubation procedure. Two windows or access points are designated on the article for protection as depicted in FIG. 1. In other embodiments, there can be three windows or access points designated as depicted in FIG. 2.

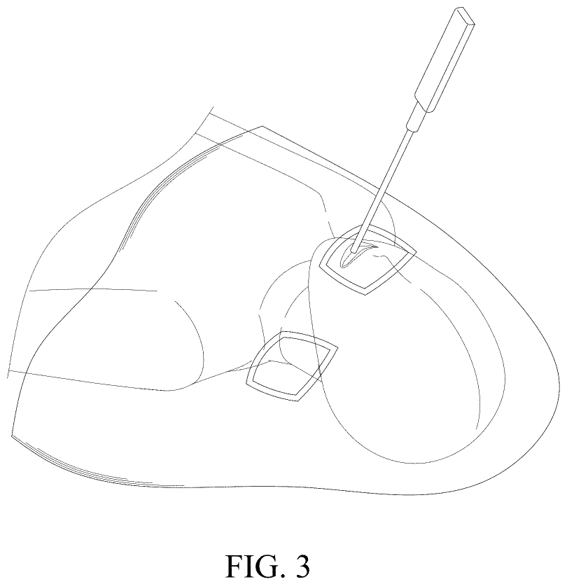

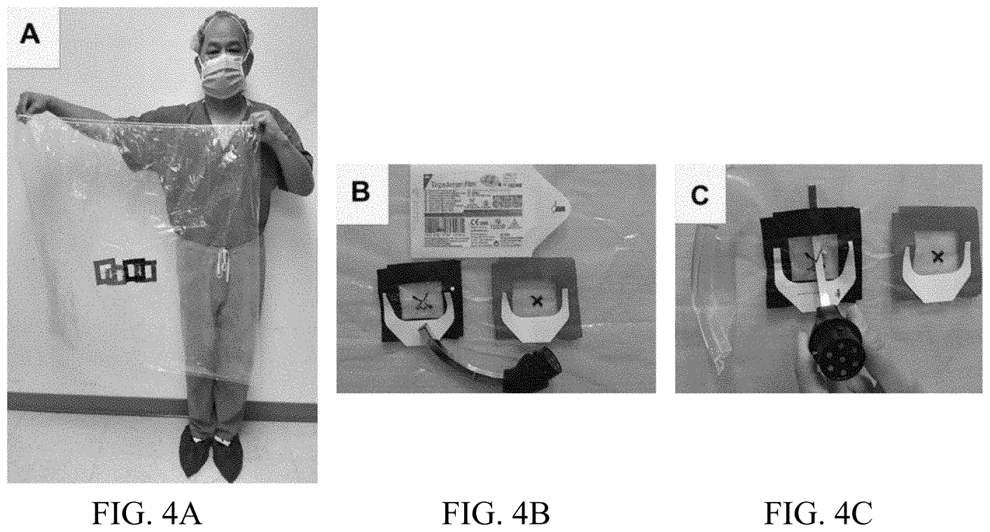

Referring now to FIG. 3, the article for protection provides an adequate coverage of the patient while allowing an intubating stylet inserting through the plastic sheet without any constrain of the clinician's movement. The plastic sheet is transparent and can be used in single or double layered. To carry out the airway management procedures with the article for protection, the plastic sheet is placed over the subject receiving the procedures to cover the entire head and upper trunk. Then, the designated windows or access points are decided to allow subsequent passage of device to be in contact with nose or mouth of the subject, while allowing adequate coverage of the plastic sheet over the entire upper trunk and head of the subject. The designated windows or access points can be marked and reinforced with colored tape in rectangles as shown in FIGS. 4A to 4C. To each layer of the sheet, a set of two crosses cut within the designated windows or access points. A larger cross at 3 cm×3 cm is designated for passage of laryngoscope blade, and the smaller cross at 2 cm×2 cm is designated for passage of endotracheal tube, as shown in FIG. 4A. In order to minimize the leak from the openings, adhesive tape is used to cover the cross areas as shown in FIG. 4B. Subsequently, a needle is used to puncture a small hole in the adhesive tape to allow the laryngoscope or endotracheal tube to pass through with ease. In order to avoid an unnecessarily large defect in the plastic sheet, the disposable video laryngoscope blade is placed under the plastic sheet prior to induction. At time of laryngoscopy, the adhesive tape is punctured with the camera module of the video laryngoscope, and then connected the laryngoscope blade to the camera underneath the plastic sheet, as shown in FIG. 4C.

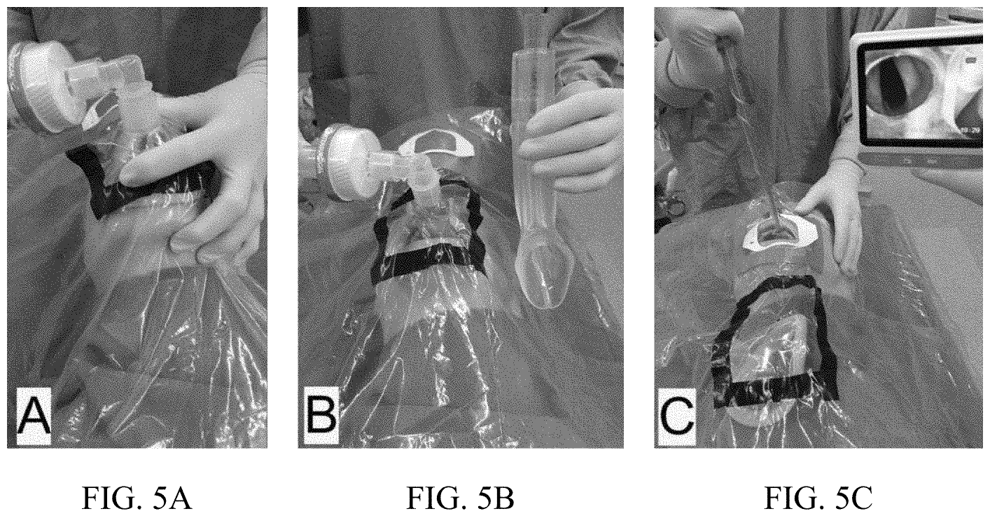

As shown in FIGS. 5A to 5C, the designated window or access points are used to allow passage of different devices. FIG. 5A shows the use of facial mask orifice connector; FIG. 5B shows the use of a laryngeal mask airway tube/connector; and FIG. 5C shows an intubating stylet or endotracheal tube. As shown in FIGS. 5A to 5C, the plastic sheet coverage does not interfere with insertion of the laryngeal mask or video-assisted intubating stylet at all. After the airway management procedure required is completed, the plastic sheet can be carefully rolled over, folded, packed and discarded.

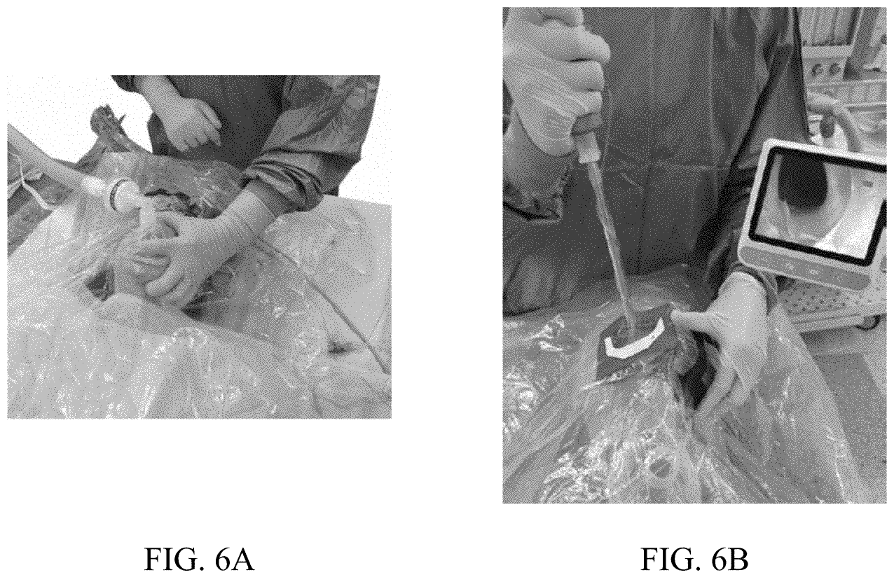

FIG. 6A shows the use of a plastic sheet as the article for protection on a patient after anesthesia is induced. The coverage of the plastic sheet does not interfere with facial mask ventilation. The facial mask ventilation can be used in its original setting without any adjustment due to the use of the article for protection.

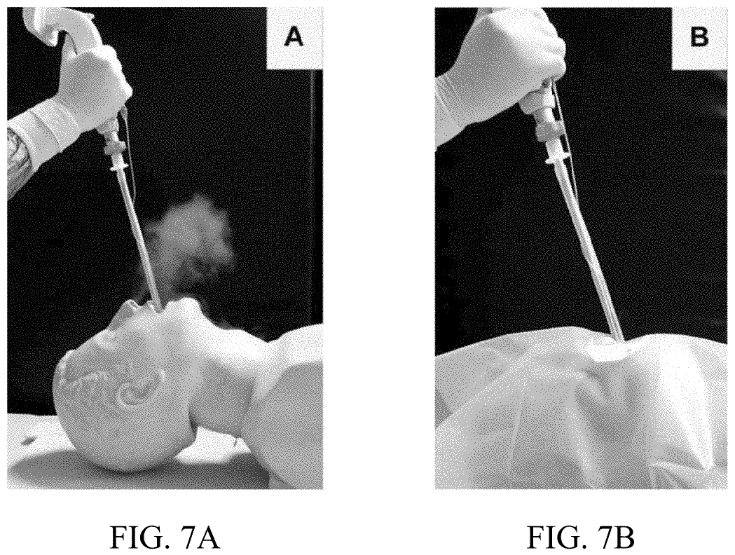

Alternatively, as shown in FIG. 6B, a video-assisted intubating stylet is used for tracheal intubation by passing through the article for protection at the designated access point. The stylet or endotracheal tube can easily pass the access point with a small aperture on the plastic sheet, and the article for protection as used does not interfere with the tracheal intubation procedure. Under the coverage of a piece of the plastic sheet, the tracheal intubation using an intubating stylet can be accomplished without any constraint to the hands of the clinicians while providing substantial protection against the secretion from accidental cough of the patient that is usually confronted during an intubation procedure. Using this intubating stylet technique, the intubator could keep a reasonable “social distance” from the patient as shown in FIG. 6B. Maintaining as much distance as possible from the subject's airway, together with protective equipment worn by the intubator and the plastic sheet barrier, reduces the chance of viral transmission from the patient's airway droplets and secretions. The use of the plastic sheet was found to not hinder the intubator's maneuvers of the video stylet. To show the effectiveness of a plastic sheet, a medical nebulizer is used to simulate aerosol production from the patient's airway. Then, the reservoir is filled with hot water, and the water is then nebulized into mist (which is composed of small droplets of water suspending in air). In some embodiments, a bag valve mask can be used to propel the mist through the mannequin's airway in order to simulate the episodes of coughing and bucking. As shown in FIG. 7A, without a plastic sheet, a visible mist was observed from the mannequin's nose and mouth. In contrast, when the mannequin's head and upper trunk were covered with a plastic sheet, the mist was not appreciated above the sheet as shown in FIG. 7B.

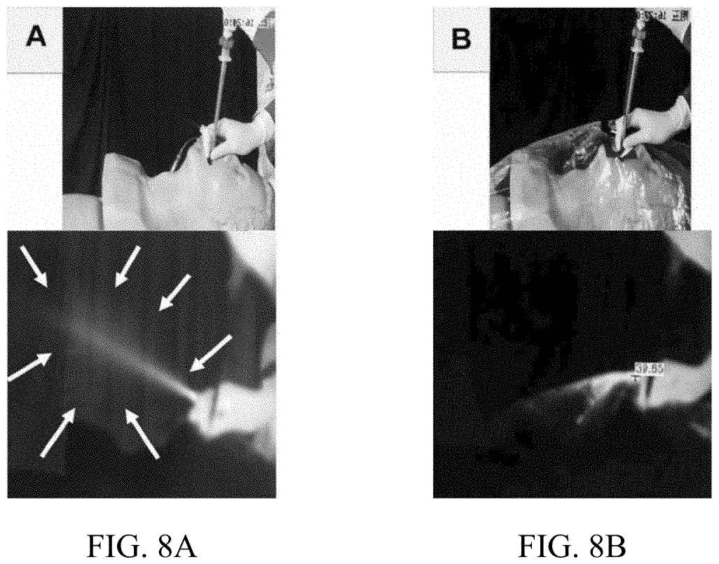

The plastic sheet is also shown to prevent spreading of larger respiratory droplets during periods of coughing, sneezing, or bucking. Hot water spray with the mannequin was used as a validation model. Hot water spray was detectable by an infrared thermography machine. FIG. 8A shows that the hot water spray was easily detected by infrared imaging, and as shown in FIG. 8B, the spray is prevented from entering the environment with application of the plastic sheet of the present disclosure.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

### REFERENCE

Canelli R., Connor C. W., Gonzalez M., Nozari A., and Ortega R. Barrier enclosure during endotracheal intubation. N. Engl. J. Med. (2020) 382:1957-1958.
Chen X., Liu Y, Gong Y, Guo X., Zuo M., Li J., Shi W., Li H., Xu X., and Mi W. Perioperative management of patients infected with the novel coronavirus: Recommendation from the Joint Task Force of the Chinese Society of Anesthesiology and the Chinese Association of Anesthesiologists. Anesthesiology (2020) 133(3):693.
Luo M., Cao S., Wei L., Tang R., Hong S., Liu R., and Wang Y. Precautions for intubating patients with COVID-19. Anesthesiology (2020) 132(6):1616-1618.
Meng L., Qiu H., Wan L., Ai Y, Xue Z., Guo Q., Deshpande R., Zhang L., Meng J., Tong C., Liu H., and Xiong L. Intubation and ventilation amid the COVID-19 outbreak: Wuhan's experience. Anesthesiology (2020) April 8: 10.

What is claimed is:

1. An article for protection on a subject in need thereof, said article comprising a covering adapted to cover at least a portion of head and upper trunk of the subject, wherein said covering includes at least one transparent portion and at least one access point designated for introducing instrument therethrough, and wherein the covering is substantially impermeable to bodily fluid of the subject.

2. The article of claim 1, wherein the at least one access point on the covering is designated at a position to allow conducting a procedure with the instrument introduced therethrough while maintaining coverage of the head and upper trunk of the subject.

3. The article of claim 1, wherein the at least one access point is located at a designated area on the covering made of a same material as other parts of the covering.

4. The article of claim 3, wherein the at least one access point is configured with a precut slit.

5. The article of claim 4, wherein the precut slit is reinforced with an additional layer of material around the slit.

6. The article of claim 4, wherein the precut slit is covered with an additional layer of flexible material substantially impermeable to the bodily fluid.

7. The article of claim 6, wherein the additional layer of flexible material is a polymeric film.

8. The article of claim 2, wherein the at least one access point is an opening configured with a cover allowing opening or closing of the access point reversibly.

9. The article of claim 2, wherein the at least one access point is a valve.

10. The article of claim 1, wherein the covering is at least one layer sheet or film made of at least one of polypropylene, polystyrene, polycarbonate, polyethylene, polyester and polypropene.

11. The article of claim 1, wherein the covering is a one-piece sheet or film integrally made with a pre-defined line of perforation configured to run across the sheet or film and through the access point.

12. A method for protecting an airway procedure, comprising:
covering the article of claim 1 over the at least a portion of head and upper trunk of the subject; and
introducing the instrument through the at least one access point of the covering.

13. The method of claim 12, further comprising making a slit at the at least one access point of the covering before introducing the instrument.

14. The method of claim 12, further comprising covering the at least one access point with an adhesive tape before introducing the instrument.
